(19) United States
(12) Patent Application Publication
(10) Pub. No.: US 2018/0021343 A1
(43) Pub. Date: Jan. 25, 2018

(54) BURN OINTMENT FOR PROMOTING TISSUE REGENERATION AND SKIN GROWTH, AND PREPARATION METHOD THEREFOR

(71) Applicant: Jin'an ZHANG, Hebei (CN)

(72) Inventors: Jin'an ZHANG, Hebei (CN); Zhuangyi ZHANG, Hebei (CN); Wenyi ZHANG, Hebei (CN)

(21) Appl. No.: 15/542,420

(22) PCT Filed: Mar. 16, 2015
(86) PCT No.: PCT/CN2015/074275

§ 371 Date: Jul. 8, 2017

(30) Foreign Application Priority Data
Jan. 21, 2015 (CN) 201510029293.0

(51) Int. Cl. A61K 31/545; A61K 31/7036; A61K 36/258; A61K 35/644; A61K 36/30; A61K 47/06; A61K 33/08; A61K 36/708; A61K 31/045; A61K 31/675; A61K 31/375; A61K 35/50; A61K 9/06; A61K 36/539

(52) U.S. Cl. CPC A61K 31/545; A61K 9/06; A61K 31/7036; A61K 36/258; A61K 35/644; A61K 36/30; A61K 36/539; A61K 33/08; A61K 36/708; A61K 31/045; A61K 31/675; A61K 31/375; A61K 35/50; A61K 47/06; A61K 2236/333; A61K 2236/53

(57) ABSTRACT

A burn ointment for promoting tissue regeneration and skin growth comprises the following raw materials (pts. wt): cephalosporin (5-15), gentamicin (5-15), Radix Ginseng (5-20), propolis (10-20), Radix Lithospermi (5-20), Radix Scutellariae (10-20), montmorillonite (10-20), Radix Et Rhizoma Rhei (6-20), human placenta tissue liquid (10-20), borneol (0.5-5), vitamin B6 (5-15), vitamin C (8-15), and ointment matrix (200). The ointment can be used for rapidly repairing local damaged tissue, accelerating wound healing and shortening the healing days of III-degree burn.

### FIELD OF THE INVENTION

The present invention relates to a burn ointment for promoting tissue regeneration and skin growth and a preparation method thereof.

### BACKGROUND OF THE INVENTION

Burn is a common disease that endangers human health and appearance. Skin injury caused by burn or new skin injury caused in the treatment process can easily cause hyperpigmentation, scar residues and other problems regarding appearance, and finally skin grafting and face-lifting will be required, resulting in aggravation of patients' pain, and expensive costs.

On the next day after burn, owing to the vesicular skin lesions and severe swelling at the burn site of patient, in accordance with the traditional therapy, skin is required to be torn off for bandaging, and should be incised by means of surgery in case of swelling exacerbation, which will aggravate the patient's pain,

In the traditional treatment methods of burn at home and abroad, after the common wound cleaning, vesicular tissues deemed as ulcer and necrotic tissues are cut off, bandaging for protection, excising eschar and grafting skin by staging, and other conservative therapies are adopted with intravenous injection of antibiotics. Several times of eschar excising and skin grafting make the patients suffer from abnormal pain and cause expensive costs, and finally, plastic surgery is required, resulting in prolonged treatment and wound being susceptible to infection. The current general exposure therapy inhibits a large number of bacterial reproduction, but at the same time also inhibits the fission and proliferation of normal cells of human bodies, thus is not beneficial to the repair and regeneration of burn wounds, and likely to cause scarring residue and prolong course of disease. Patients will overcome “Three Difficulties” to escape with bare life.

The Chinese patent ZL95118978.6 is an early research result of the inventor, which discloses an anti-inflammatory and analgesic burn ointment. Owing to the integration of antibiotics and traditional Chinese medicines, the burn ointment has strong anti-infective effect and good analgesic effect, and can shorten the wound healing period and effectively reduce the incidence of scars. The healing time for deep II degree burn wounds under the treatment of the ointment of ZL95118978.6 is 4 to 13 days, which is significantly shortened compared with the control group of “MEBO” moisture exposed burn ointment. But the healing time for III degree burn wound under the treatment of the ointment of ZL95118978.6 is 21 to 30 days, which is still long.

### BRIEF SUMMARY OF THE INVENTION

In view of the above technical problems, the object of the present invention is to provide a burn ointment for promoting tissue regeneration and skin growth, which can effectively shorten the healing time of III degree burn wound without scarring and effectively alleviate the patients' pain, and to provide a preparation method thereof The ointment can solve the world-class problems of “no scarring for II degree burn and no skin grafting for III degree burn”, namely surgery skin grafting is replaced with tissue regeneration and skin growth by means of ointment application.

In order to achieve the above object, the present invention adopts the following technical solution:

The burn ointment for promoting tissue regeneration and skin growth comprises the following components in parts by weight: 5-15 parts of cephalosporin, 5-15 parts of gentamicin, 5-20 parts of radix ginseng, 10-20 parts of propolis, 5-20 parts of radix lithospermi, 10-20 parts of radix scutellariae, 10-20 parts of montmorillonite, 6-20 parts of radix et rhizoma rhei, 10-20 parts of human placenta tissue liquid, 0.5-5 parts of borneol, 5-15 parts of vitamin B6, 8-15 parts of vitamin C, and 200 parts of ointment matrix.

Preferably, cephalosporin is cefoperazone sodium. For special patients, other antibiotics can be selected, according to their bacterial culture drug-susceptibility testing results.

Preferably, the ointment matrix is Vaseline or W/O type matrix.

The human placenta tissue liquid is commercially available, such as human placenta tissue liquid with the approval number of “State Food and Drug Administration Approval Number S13020002” and produced by Handan Kangye Pharmaceutical Co., Ltd.

The present invention also provides the preparation method for the burn ointment for promoting tissue regeneration and skin growth, which comprises the following steps of:
(1) weighing radix ginseng, propolis, radix lithospermi, radix Scutellariae and radix et rhizoma rhei by above weight, infiltrating in 75% alcohol for 18-24 hours, drying, putting into the drying oven, drying at 30-40° C., crushing, screening with 120 mesh sieves for later use;
(2) weighing montmorillonite by above weight, grinding, screening with 120 mesh sieves, adding borneol, and grinding for later use; and
(3) weighing cephalosporin, gentamicin, vitamin B6 and vitamin C by above weight, mixing uniformly, adding the mixture obtained in the steps (1) and (2), mixing uniformly, adding the human placenta tissue liquid and the molten ointment matrix, and grinding to uniform.

### BRIEF DESCRIPTION OF THE INVENTION

When the ointment of the present invention is used for treatment, the exposure therapy is adopted. After the burn wounds are normally cleaned, the blister is punctured to release water, without tearing off ulcer and the necrotic tissues mistaken in the traditional therapy. The burn ointment is uniformly applied to wounds, on the next day, the original wounds are applied with the burn ointment again (the original ointment is not necessarily removed), and the ointment is repeatedly applied until medicinal eschars occur, and the original appearance is restored after eschars are dropped and wound is cured.

According to the present invention, the pharmaceutical components are reasonably mixed, thus the burn ointment of the present invention can promote tissue regeneration and skin growth while effectively killing bacteria left or reproduced in the damaged skin tissues and eliminating acute infection pathogeny. The ointment has high transdermal absorbance and obviously reduces wound exudate, the local damaged tissues can be quickly repaired, the healing of the wounds is accelerated, thereby the healing time of III degree burn wound is significantly shortened, the regeneration of granulation tissues is effectively avoided, the growth of large fibroblasts is inhibited and the formation of scars is prevented. In addition, the ointment of the present invention also has a good analgesic effect, greatly relieves the pain of the patients caused by burn, and also effectively avoids negative physiological and pathological effects of the patient caused by burn pain.

The positive effects of the medicament of the present invention are confirmed from the following clinical validation data.

I. The Choice of Burn Cases:

Patients with III degree burn in accordance with Surgery written by HUANG Jiasi are divided into two groups by double-blind random approach. For the experimental group, the medicament of the present invention is used, and “MEBO” moisture exposure burn ointment is used for the control group.

II. Evaluation Standard of Curative Effect

1. Evaluation standard for wound infection

Wound infection as per Pruitt (1979)

2. Evaluation standard for wound healing

Standard as per Surgery written by HUANG Jiasi

3. Evaluation standard for scar healing

Standard as per Surgery written by HUANG Jiasi

4. Evaluation standard for wound pain

Patient who feels unbearably pain after 15 minutes and requires the use of analgesics is classified as a positive statistical object.

III. Clinical Observation Results

TABLE 1
Comparison of incidence rate of wound infection

| Groups | Total number of | Number of | Infection rate | P |
|---|---|---|---|---|
| Experimental | 180 | 0 | 0 | |
| Control group | 120 | 3 | 2.50% | <0.01 |

TABLE 2
The comparison of the incidence rate of burn scars

| Groups | Total number of | Number of scars | Scar rate | P |
|---|---|---|---|---|
| Experimental | 240 | 0 | 0 | |
| Control group | 240 | 128 | 53 | <0.05 |

TABLE 3
The comparison of skin-grafting rate of wounds

| Groups | Total number of | Number of skin | Skin-grafting rate | P |
|---|---|---|---|---|
| Experimental | 180 | 0 | 0 | |
| Control group | 120 | 2 | 1.57% | <0.05 |

TABLE 4
The comparison of the incidence rate of wound pain

| Groups | Total number of | Number of pain | Pain rate | P |
|---|---|---|---|---|
| Experimental | 180 | 0 | 0 | |
| Control group | 120 | 38 | 31.6 | <0.01 |

Comparison between the present invention and Chinese patent ZL95118978.6 with respect to the healing time of III degree burn wounds is shown in Table 5 (wherein, the ointment of the present invention is used for experimental group, and ointment of ZL95118978.6 is used for the control group).

TABLE 5
Comparison of healing time of III degree wounds.

| | | Area group | | |
|---|---|---|---|---|
| | | Healing time | | |
| | | Group of less | Group of | Group of |
| Age group | | than 10% | 11-20% | 21-51% |
| Group of less | Experimental | 6.50 ± 2.01 | 6.50 ± 3.10 | 6.85 ± 3.90 |
| than 20 years | group | | | |
| old | Control group | 18.33 ± 3.51 | 19.61 ± 3.39 | 20.48 ± 3.82 |
| | P | <0.05 | <0.05 | <0.05 |
| Group of 20-40 | Experimental | 7.20 ± 3.12 | 6.12 ± 3.12 | 7.60 ± 3.11 |
| years old | group | | | |
| | Control group | 20.11 ± 3.73 | 20.31 ± 3.11 | 21.45 ± 3.43 |
| | P | <0.05 | <0.05 | <0.05 |
| Group of more | Experimental | 6.0 ± 3.10 | 6.5 ± 2.26 | |
| than 40 years | group | | | |
| old | Control group | 30.41 ± 3.24 | 31.31 ± 2.78 | |
| | P | <0.05 | <0.05 | |

Typical Cases

1. ZHANG Ansheng, male, 38 years old, resided in Yan Village, Taoyuan Township, Cao County, Shandong Province.

Nov. 26, 2014, 15:30

Chief complaints: face burn for half a day

At 11 am on that day, face is burned due to the explosion of the pressure cooker during home cooking. The patient immediately went to Burn Department of First Hospital of Hebei Medical University for liquid medicine spraying, but the patient could not bear the pain and went to see the reputed doctor.

Examination: face flushing, blister, III degree burn, restless, and the patient immediately fall asleep with snoring after application of the ointment of the present invention.

Treatment: conventional disinfection, the burn ointment of the present invention was externally applied and washed off completely on the 5th day, and the face of the patient is as usual as ordinary people.

2. TAN Jian, male, 28 years old, active army in Security Department of Ordnance Engineering College of the Chinese People's Liberation Army.

May 25, 2007

Chief complaint: face burn for 6 days

Face and left arm were burn 6 days ago by setting off fireworks. The patient was conveyed to Burn Department of Peace Hospital of the People's Liberation Army for hospitalization. The patient's face was disfigured with eschar, and was swollen like a bucket, belonging to III degree burn. The director of Burn Department prepared skin grafting on the 6th day, and informed the patient of the risk of disfigurement, hence, the patient went to see a reputed doctor. After the application of the ointment of the present invention, burn is cured after 10 days with no skin grafting, and there is no scar and hyperpigmentation on the face, which is as usual as ordinary people.

3. ZHANG Risheng, male, 58 years old. He was burn in accident fire during the maintenance of gas pipelines on Dec. 1, 2014, with III degree burn for the face, neck and hands. The moisture ointment was externally applied immediately, face and hands were severely swelling on the next day, and most of face skin begun to peel, with unbearable pain. The ointment of the present invention was externally applied without transfusion and hospitalization and the wound was cured after 6 days without scars, and the patient looked as usual.

### Embodiment

### EXAMPLE 1

The example comprises the following components by weight: 15 kg of cefoperazone sodium, 15 kg of gentamicin, 15 kg of vitamin C, 15 kg of vitamin B6, 20 kg of radix ginseng, 20 kg of radix scutellariae, 20 kg of propolis, 20 kg of radix lithospermi, 6 kg of radix et rhizoma rhei, 20 kg of montmorillonite, 10 kg of human placenta tissue liquid, 5 kg of borneol, and 200 kg of ointment matrix.

The preparation method of the embodiment comprises the following steps of:
(1) weighing radix ginseng, propolis, radix lithospermi, radix scutellariae and radix et rhizoma rhei by above weight, infiltrating in 75% alcohol for 18-24 hours, drying, putting into the drying oven, drying at 30-40° C., crushing, screening with 120 mesh sieve for later use;
(2) weighing montmorillonite by above weight, grinding, screening with120 mesh sieve, adding borneol, and grinding for later use; and
(3) weighing cephalosporin, gentamicin, vitamin B6 and vitamin C, mixing uniformly, adding the mixture obtained in the steps (1) and (2), mixing uniformly, adding the human placenta tissue liquid and the molten ointment matrix, and grinding to uniform.

Grinding montmorillonite for crushing, screening with 120 mesh sieve, adding borneol, uniformly grinding for later use;

mixing cefoperazone, gentamicin, compound vitamins C, grinding to fine powder, adding Chinese herbal medicine powder and borneol powder, mixing uniformly, finally, adding the molten sesame oil, and fully and uniformly grinding.

The preparation methods of the following six examples are the same as that of the example 1, except for difference in the ratios of medicinal components, but the curative effect is substantially the same.

| | Example | | | | | |
|---|---|---|---|---|---|---|
| Component (g) | 2 | 3 | 4 | 5 | 6 | 7 |
| Cefoperazone sodium | 5 | 8 | 10 | 15 | 12 | 10 |
| Gentamicin | 5 | 8 | 15 | 5 | 15 | 10 |
| Human placenta tissue liquid | 15 | 12 | 10 | 20 | 18 | 10 |
| Vitamin C | 10 | 15 | 11 | 8 | 12 | 12 |
| Radix Ginseng | 5 | 15 | 10 | 8 | 12 | 10 |
| Vitamin B6 | 5 | 8 | 10 | 15 | 12 | 6 |
| Radix Et Rhizoma Rhei | 10 | 8 | 15 | 20 | 12 | 6 |
| Radix Scutellariae | 10 | 20 | 15 | 20 | 12 | 10 |
| Propolis | 10 | 20 | 15 | 10 | 12 | 10 |
| Radix lithospermi | 10 | 10 | 15 | 10 | 5 | 10 |
| Montmorillonite | 10 | 8 | 15 | 20 | 12 | 10 |
| Borneol | 0.5 | 3 | 5 | 1 | 4 | 0.5 |
| Vaseline | 200 | 200 | | | 200 | 200 |
| W/O type matrix | | | 200 | 200 | | |

1. A burn ointment for promoting tissue regeneration and skin growth, characterized in that_{;} it comprises the following components by weight: 5-15 parts of cephalosporin, 5-15 parts of gentamicin, 5-20 parts of radix ginseng, 10-20 parts of propolis, 5-20 parts of radix lithospermi, 10-20 parts of radix scutellariae, 10-20 parts of montmorillonite, 6-20 parts of radix et rhizoma rhei, 10-20 parts of human placenta tissue liquid, 0.5-5 parts of borneol, 5-15 parts of vitamin B6, 8-15 parts of vitamin C, and 200 parts of ointment matrix.

2. The burn ointment for promoting tissue regeneration and skin growth according to claim 1, wherein the cephalosporin is cefoperazone sodium.

3. The burn ointment for promoting tissue regeneration and skin growth according to claim 1, wherein the ointment matrix is Vaseline or W/O type matrix.

4. A method for preparing a burn ointment according to claim 1, for promoting tissue regeneration and skin growth, said method characterized in that it comprises the following steps:
(1) preparing radix ginseng, propolis, radix lithospermi, radix Scutellariae and radix et rhizoma rhei in weight parts recited in claim 1, infiltrating in 75% alcohol for 18-24 hours, drying at 30-40° C., crushing, and screening with a 120 mesh sieve;
(2) preparing montmorillonite in weight parts recited in claim 1, grinding, screening with a 120 mesh sieve, adding borneol, and grinding; and
(3) preparing cephalosporin, gentamicin, vitamin B6 and vitamin C in weight parts recited in claim 1, mixing uniformly, adding the mixture obtained in steps (1) and (2), mixing uniformly, adding the human placenta tissue liquid and the molten ointment matrix, and grinding to uniform.
